(19) United States
(12) Patent Application Publication
(10) Pub. No.: US 2009/0082568 A1
(43) Pub. Date: Mar. 26, 2009

(54) ACCELERATED REDUCTION OF ORGANIC SUBSTANCES WITH BORANES

(75) Inventor: Elizabeth Burkhardt, Bridgeville, PA (US)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/859,390

(22) Filed: Sep. 21, 2007

(51) Int. Cl. C07D 213/02; C07C 209/28; C07C 27/04; C07F 5/02; C07D 207/00

(52) U.S. Cl. 546/13; 548/110; 564/489; 568/6; 568/884

(57) ABSTRACT

In a process for the accelerated reduction of organic substrates, selected from the group consisting of ester, amides, nitriles, acids, ketones, imines or mixtures thereof, the substrates are reacted with an amine borane, sulfide borane or ether borane complex as a borane source in the presence of organic accelerator compounds containing both Lewis acidic and Lewis basic sites in their structure, of which the Lewis acidic site can coordinate with the carbonyl or nitrile or imine group of the substrate and the Lewis basic site can coordinate with the borane.

### FIELD OF THE INVENTION

The present invention relates to new methods to accelerate the reduction of organic substrates like esters and amides using boranes like amine boranes with catalytic amounts of additives.

### BACKGROUND OF THE INVENTION

The reduction of organic substrates, e.g. an ester, acid or ketone to an alcohol and an amide, nitrile or imide to an amine is a key transformation for the development of pharmaceutical drugs such as antibacterials, HIV inhibitors and ocular hypertension drugs. These transformations are difficult to complete selectively in the presence of other sensitive reducible functional groups. The introduction of new methods for the reduction of these organic substrates, especially of esters and amides is highly desirable.

Amine borane complexes are very stable borane sources. The borane complexes of amines are easily used on a large scale but generally less reactive than borane complexes of ethers or sulfides. Some amine boranes are even stable to aqueous solution over extended periods of time. Their applications in organic synthesis have been limited due to their low reactivity toward functional groups. In contrast to other more reactive borane complexes such as borane tetrahydrofuran (BTHF) or dimethylsulfide borane (DMSB), acidic conditions or elevated temperatures are normally required in reductions with amine boranes. Pyridine borane and trimethylamine borane are often insufficiently reactive to accomplish the amide reduction. Borane derivatives of dialkylanilines and sterically hindered amines are significantly more reactive than other amine boranes but still require prolonged heating at elevated temperatures to drive the amide reduction to completion, see Brown, H. C.; Kanth, J. V. B.; Zaidlewicz, M. J. Org. Chem. 1998, 63(15), 5154-5163. Salunkhe, A. M.; Burkhardt, E. R. Tetrahedron Letters 1997, 38(9), 1519; Brown, H. C.; Kanth, J. V. B.; Dalvi, P. V.; Zaidiewicz, M. J. Org. Chem. 1999, 64(17), 6263-6274. Kanth, J. V. B. Aldrichimica Acta 2002, 35, 57. Burkhardt and Salunkhe reported that N,N-diethylaniline borane (DEANB) efficiently reduced a variety of functional groups such as aldehydes, ketones, carboxylic acids, esters and tertiary amides at elevated temperature. Esters and hindered ketones required extensive reaction time at reflux in THF to drive the reaction to completion. These examples demonstrated lower reactivity of DEANB versus BTHF and DMSB, see Bonnat, M.; Hercouet, A.; Le Corre, M. Synthetic Communications 1991, 21(15-16), 1579-82. However, due to the thermal ether cleavage of BTHF and the stench of DMSB, high volume use of these borane reagents for ester and amide reductions is limited.

The reduction of the ester functionality with borane complexes requires harsh conditions, generally requiring refluxing conditions to effectively push the reduction to completion. Several examples exist using BTHF or DMSB for this purpose, see Sessler, J. L. et al. Inorg. Chem. 1993, 32, 3175 and Brown, H. C.; Choi, Y. M.; Narasimhan, S. J. Org. Chem. 1982, 47(16), 3153-63. When DMSB is used, the dimethyl sulfide is usually distilled from the refluxing solution to drive the reduction to completion. For example, selective reduction of one ester of L-maleic acid dimethylester using DMSB successfully produced 3(S)-4-dihydroxybutyric acid methyl ester. Amine boranes generally do not reduce the ester functionality. However, due to the thermal ether cleavage of BTHF and the stench of DMSB, high volume use of these borane reagents is limited. Clearly, new methods must be developed for ester reductions.

Salunkhe and Burkhardt (see above) demonstrated that DEANB is a very effective reducing agent of prochiral ketones in the presence of an oxazaborolidine catalyst. With MeCBS the reduction was complete in less than 2 hours at ambient temperature whereas without catalyst the ketone reduction was 56% complete in 27 h. The acceleration of ketone reduction by BTHF with an oxazaborolidine catalyst has been studied by Jockel, H.; Schmidt, R.; Jope, H.; Schmalz, H-G. J. Chem. Soc. Perkin Trans. 2, 2000, 69. Schmidt, R.; Jockel, H.; Schmalz, H-G; Jope, H. J. Chem. Soc. Perkin Trans. 2, 1997, 2725. Since the reduction of amides and esters does not provide a new chiral center, it is not intuitively obvious to try the available chiral oxazaborolidine catalyst in this type of reduction.

### SUMMARY OF THE INVENTION

The object of the present invention is to provide new methods to accelerate the reduction of organic substrates like esters and amides using boranes, e.g. amine boranes, with catalytic amounts of additives.

The object is achieved by a process for the accelerated reduction of organic substrates, selected from the group consisting of esters, amides, nitriles, acids, ketones, imines or mixtures thereof, by reacting with an amine borane, sulfide borane or ether borane complex as a borane source in the presence of organic accelerator compounds containing both Lewis acidic and Lewis basic sites in their structure, of which the Lewis acidic site can coordinate with the carbonyl or nitrile or imine group of the substrate and the Lewis basic site can coordinate with the borane.

Preferably, esters, acids and ketones are reduced to give alcohols, and amides, nitriles and imines are reduced to give amines.

Preferably, the amine borane, the sulfide borane and the ether borane are derived from amines, sulfides and ethers which conform to the formulae

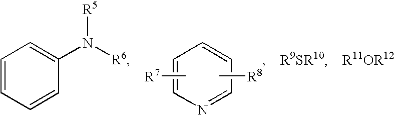

wherein R⁵-R¹² independently are C₁₋₆-alkyl, phenyl, or in which each two of R⁵ and R⁶, R⁹ and R¹⁰, R¹¹ and R¹² independently can together form an C₄₋₆-alkylene group, and R⁵-R¹² can be substituted by halogen and R⁷ and R⁸ can also be hydrogen.

In the specification and claims, “alkyl” and “alkylene” can be linear or branched alkyl or alkylene.

Preferably, the amine borane is a tertiary amine borane, especially N,N-diethylaniline (DEANB), the sulfide borane is dimethylsulfide borane (DMSB), and the ether borane is borane tetrahydrofuran (BTHF) or borane 2-methyl tetrahydrofuran.

Preferably, the organic substrate contains 4 to 30 carbon atoms.

Preferably, the organic substrate contains one or more of alkyl, aryl, aralkyl, alkaryl, heterocycloalkyl, and heteroaryl groups besides the ester, amide, nitrile, acid, keto or imino functional group. The substrate may contain other functional groups not reduced by borane such as alkoxy, halo, nitro, sulfonamide or the groups can be tri- or tetrasubstituted alkene that reacts slower with borane than the catalyzed reduction.

Preferably, the esters, amides, nitriles, acids, ketones and imines conform to the formulae R¹—C(═O)—OR² R¹—C(═O)—NR³R⁴ R¹—CN R¹—C(═O)OH R¹—C(═O)—R² R¹R²C═NH R¹R²C═NR³

wherein
R¹-R⁴ independently are C₁₋₁₂-alkyl, C₆₋₁₂-aryl, C₇₋₁₂-aralkyl, C₇₋₁₂ alkaryl, which can be substituted with other functional groups as described above.

Preferably, the organic accelerator compound contains a structural element of the formula N—B or is an oxazaborolidine or cyclic compound containing a structural element of the formula N—B—O where N— and O— are connected by a carbon chain.

The organic accelerator compound is preferably derived from secondary amino alcohol via reaction with e.g. boranes or borates. The aminoalcohol fragment may be attached to a polymer chain.

Preferably, the organic accelerator compound is a spiroborate compound containing a structural element of one of the following formulae of which only the core structure is shown but not the residues like alkyl or alkylene chains

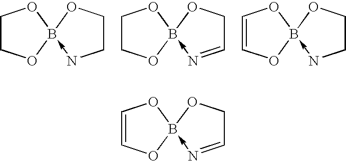

in which the rings can contain 5, 6 or 7 elements. The further elements not shown are preferably carbon-based elements.

Preferably, the organic accelerator compound has one of the following general formulae

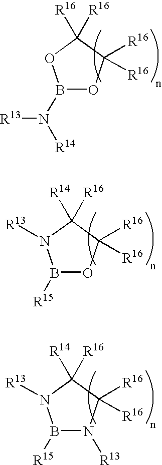

wherein
R¹³, R¹⁴, R¹⁵, R¹⁶ at each position independently are hydrogen, C₁₋₁₂-alkyl, C₁₋₁₂-aryl, C₇₋₁₂-aralkyl, C₇₋₁₂-alkaryl, wherein R¹³ and R¹⁴ or wherein R¹³ and R¹⁵ can together form a cyclic residue, with the proviso that not more than 4 residues R¹⁶ are different from hydrogen,
n is 1, 2 or 3

Preferably, the oxazaborolidine compound is selected from the group consisting of

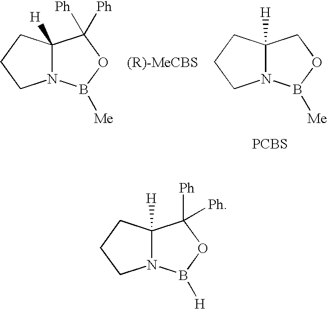

Preferably, the spiroborate compound is selected from the group consisting of

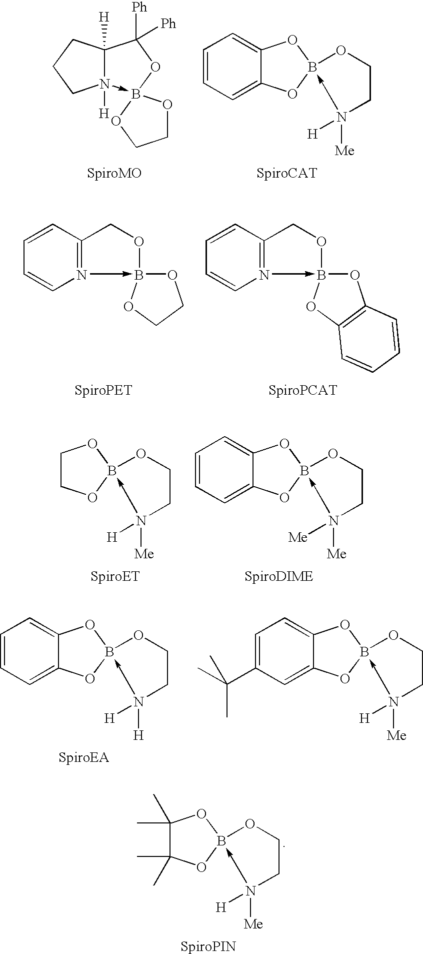

Preferably, the amount of accelerator compound, based on the amine borane, sulfide borane or ether borane is 0.01 to 100 mol-%.

The object is furthermore achieved by a composition for the accelerated reduction of organic substrates, selected from the group consisting of esters, amides, nitriles, acids, ketones, imines or mixtures thereof, comprising at least one amine borane, sulfide borane or ether borane complex as a borane source and at least one organic accelerator compound containing both Lewis acidic and Lewis basic sites in their structure, of which the Lewis acidic site can coordinate with the carbonyl or nitrile or imino group of a substrate and the Lewis basic site can coordinate with the borane.

Furthermore, the object is achieved by an organic accelerator compound as defined above in the formulae.

The inventors have found that the reduction of organic substrates selected from esters, amides, nitriles, acids, ketones, imines, preferably esters and amides, especially esters and tertiary amides by reacting with a borane source can be accelerated by organic accelerator compounds which contain in the same molecule both Lewis acidic and Lewis basic sites. The Lewis acidic site is such that it can coordinate with the carbonyl or nitrile or imino group of the substrate, and the Lewis basic site is such that it can coordinate with the borane. A person skilled in the art will immediately recognize whether a Lewis acidic site and Lewis basic site fulfils these requirements.

Without being bound by any theory, the additives are envisioned to increase the reaction rate by two divergent mechanisms, a) coordination of a Lewis acid to the carbonyl of the substrate to increase the carbocation (electrophilic) character of the carbon, or b) dynamic equilibrium of the borane coordination to the additive to facilitate interaction of the substrate with borane. More detailed oxazaborolidine additives are envisioned to increase the reaction rate by two convergent mechanisms, a) coordination of the carbonyl of the substrate to a Lewis acidic boron to increase the carbocation (electrophilic) character of the carbon, coupled with b) dynamic equilibrium of the borane coordination to the Lewis basic nitrogen center of the additive to facilitate proximal interaction with the substrate with borane. Other acceleration agents with both a Lewis acidic site and a Lewis basic site also are anticipated to assist the carbonyl reduction by a mechanism of bringing the activated carbonyl and the borane into close proximity to thereby lower the activation energy of the reduction.

The process can be carried out in presence or in the absence of a solvent.

Accordingly, esters of the formula,

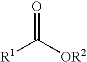

and amides of the formula,

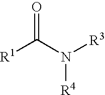

can be preferably effectively reduced with borane, complexed by amines, sulfides or ethers of the formula,

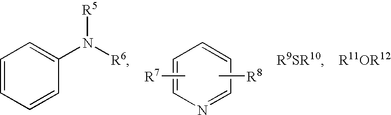

by the addition of catalytic amounts of the rate acceleration agents. These rate acceleration agents can be of a structure containing both Lewis acidic and Lewis basic sites, such as more preferably

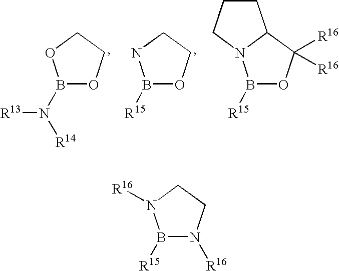

with the above meanings for R¹³-R¹⁶ such that the carbonyl of the substrate (amine or ester) can coordinate (Lewis acidic site) and the borane can coordinate (Lewis basic site) proximal to the activated carbonyl.

The acceleration agent can be mixed with an organic substrate, e.g. the ester or amide prior to addition of the (amine) borane or combined with the (amine) borane prior to addition to the substrate.

Furthermore, the (amine) borane and acceleration agent can be combined into a formulation to facilitate the large-scale use of the combination (formulation mixture) for the reduction of organic substrates, e.g. esters and amides. The amount of accelerator is preferably 0.01 to 20 mol-%, more preferably 0.05 to 10 mol-%.

Another embodiment of the present invention are solutions comprising a borane complex as described, at least one of the acceleration agents (as defined) and optionally at least one solvent.

The new composition of (amine) borane (e.g. N,N-diethylaniline, 2,6-lutidine, 2-chloropyridine) with accelerator additive and preferred process of ester and amide (functional groups) reduction of the present invention can preferably be employed for transformations of esters to alcohols and amides to amines (nitrile to amine).

### DETAILED DESCRIPTION OF THE INVENTION

In a preferred embodiment of the present invention the new process comprises the step of contacting an (amine) borane, an acceleration agent (catalyst) and organic substrate, e.g. an ester or amide substrate in a reaction vessel. The reaction could also be carried out easily in a continuous process.

A preferred embodiment of the present invention is where the (amine) borane and an acceleration agent (catalyst) are combined then added to an organic substrate, e.g. ester or amide substrate in a reaction vessel at the desired temperature. The formulations of the present invention generally contain the new composition of (amine) borane of the above formula with concentrations of acceleration agent between 0.0005 and 0.5 mol per mole of (amine) borane, preferably between 0.0005 and 0.2 mol per mole of (amine) borane, more preferably between 0.001 and 0.1 mol per mole of (amine) borane.

A preferred embodiment of the process of the present invention comprises the addition of an acceleration agent to the organic substrate, e.g. ester or amide prior to addition of (amine) borane to the reaction.

Another preferred embodiment of the process of the present invention comprises the addition of an (amine) borane containing the acceleration agent to the organic substrate, e.g. ester or amide in a solvent. Of course, one or more other solvents with lower complexing ability to borane than the recommended amine may also be present. Suitable solvents for the reaction solutions of the present invention are those in which the (amine) borane complexes have a high solubility. Examples are ethers like diethyl ether, 1,2-dimethoxyethane, tetrahydrofuran or 2-methyltetrahydrofuran, sulfides like dimethyl sulfide or 1,6-thioxane (these sulfides also act as borane complexing agent) and hydrocarbons like pentane, hexane(s), heptane(s), cyclohexane, toluene or xylenes. Preferred solvents for the solutions of the (amine) borane-acceleration agent formulation are tetrahydrofuran, 2-methyltetrahydrofuran, dimethyl sulfide, 1,6-thioxane, toluene, hexane(s), heptane(s) or cyclohexane, most preferred are tetrahydrofuran, 2-methyltetrahydrofuran, and toluene.

The process of the present invention can generally be carried out at a temperature of from 0 to +150° C., preferably of from 10 to 110° C. and more preferably from 20 to 85° C.

The pressure is typically ambient pressure, preferably in the range of from 0.1 to 10 bar, especially 0.5 to 2.5 bar.

Those skilled in the art will appreciate that the invention described herein is subject to variations and modifications other than those specifically described herein. It is to be understood that the invention includes all such variations and modifications. The invention also includes all of the steps, features, compounds and compositions referred to or indicated in this specification, individually or collectively, and any and all combinations of any two or more of said steps or features.

The following examples illustrate the present invention without limitation of the same. The described examples do not supersede the generality of the invention as described above.

### EXAMPLES

In the following, procedural examples, preparation and test examples as well as reduction examples are given.

### Procedural Examples

Some reactions were carried out in the stainless steel 1 liter pressure reactor equipped with a ASI/Mettler React-IR for analysis. Before use, the reactor was cleaned and purged with nitrogen. The React-IR was set-up and calibrated according to the recommended manufacturer procedure before acquiring spectra.

Other reactions were conducted in typical oven-dried glassware under nitrogen. Samples were withdrawn, quenched and analyzed by FT-IR or GC as described in detail below.

### Procedural Example 1

### Reduction of Esters and Amides at 50° C.

The reactor was charged with a solution of 200 mLs of dry TH F and 0.1 mol ester or amide and heated to 50° C. under 20 psi nitrogen pressure with a back-pressure-regulator (BPR) set at 25 psi. DEANB (mols dependent on substrate) was fed subsurface at 30 psi over 1 hr maintaining a reaction temperature of 50° C. Completion of the reaction was determined by disappearance of the carbonyl stretch (wavenumber dependent on substrate). After all data was collected and analyzed, the reaction was quenched with 50 mLs of MeOH at 7 to 10° C.

### Procedural Example 2

### Reduction of Esters and Amides at 85° C.

Reductions at 85° C. were carried out in a pressure vessel with 30 psi of nitrogen pressure, BPR of 35 psi, and a feed pressure of 40 psi. Concentration and addition time were the same as in procedural example 1.

### Procedural Example 3

### Reduction of Substrates in Glassware at 50° C.

Smaller scale screening reactions were completed in glassware. A 100 mL three-neck round bottom flask (clean oven-dried) fitted with condenser to N₂ bubbler, septa and thermocouple was charged with 0.05 mol ethylbutyrate or ethylbenzoate, 10 mLs THF and stirred for 15 minutes. After heating the flask to 50° C., a mixture of 0.05 mols of DEANB (with or without additive) was slowly added to the flask. To determine reduction time, 1 mL samples were hydrolyzed with 0.5 mL methanol and FT-IR spectrometry was used to monitor the disappearance of the carbonyl stretch (1734-1654 cm⁻¹ dependent on substrate).

### Procedural Example 4

### Reduction of Substrates in Glassware at 20° C.

Smaller scale screening reactions were completed in glassware. A 100 mL three-neck round bottom flask (clean oven-dried) fitted with condenser to N₂ bubbler, septa and thermocouple was charged with 0.05 mol ethylbutyrate or ethylbenzoate, 10 mLs THF and stirred for 15 minutes at ambient temperature, 20° C. A mixture of 0.05 mols of DEANB (with or without additive) was slowly added to the flask. To determine reduction time, 1 mL samples were hydrolyzed with 0.5 mL methanol and FT-IR spectrometry was used to monitor the disappearance of the carbonyl stretch (1734-1654 cm⁻¹ dependent on substrate).

Ratio of 1 equivalent of Substrate to DEANB:

| Substrate | Equivalents of DEANB |
|---|---|
| Ethylbutyrate | 1 |
| Ethylbenzoate | 1 |
| N,N-dimethylacetylamide | 1.67 |
| N-methylpropionamide | 2 |
| n-butyramide | 2.33 |
| Acetophenone | 1 |
| Propionic acid | 1.33 |
| n-heptane nitrile | 1.33 |

### Preparation (P) and Test (T) of the Accelerators

### Example P1

### 2-(methylamino)ethanol catechol spiroborate (SpiroCAT) via CATB and 2-(methylamino)ethanol

### CAS Name: Ethanamine, 2-(1,3,2-benzodioxaborol-2-yloxy)-N-methyl-

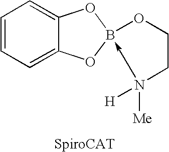

A clean dry 200 mL 3-neck round bottom flask was purged with nitrogen and charged with 0.084 mols (10 g) of catecholborane (CATB) and 100 mL toluene. The flask was cooled with and ice-water bath and 0.084 mols (6.3 g) of 2-(methylamino)ethanol was fed over 1 hr and 30 mins. The clear solution became turbid and eventually become a thick white slurry (difficult to stir with magnetic stir-bar). Reaction temperature increased from 1.8 to 7.0° C. and 0.043 mols (1.05 L) of H₂ was evolved during addition. The resulting slurry was stirred at room temperature overnight before vacuum filtering and drying overnight to yield 15.0 g of a white powder (92.7% yield). The product was difficult to obtain a representative ¹¹B-NMR and ¹H-NMR spectra due to its insolubility in the deuterated solvents tested (DMSO, DMS, chloroform, THF, benzene).

¹¹B-NMR (300 MHz, d-tetrachloroethane) 7.9 ppm.

¹H-NMR (300 MHz, d-tetrachloroethane) □ ppm: 2.39 (s, H3), 2.85 (t, H2), 3.59 (t, H2), 6.56 (H2), 6.65 (H2).

### Example TI

### Ester Reduction with SpiroCAT

A standard reduction of 0.05 mols ethylbutyrate in THF with 0.05 mols DEANB and 10 mol % SpiroCAT at room temperature was complete in 4.5 hrs. Reaction was monitored by FT-IR, ethylbutyrate carbonyl stretch at 1734 cm⁻¹.

### Example P2

### 2-(methylamino)ethanol catechol spiroborate (SpiroCAT) via Catechol, IPB and 2-(methylamino)ethanol

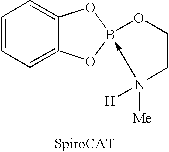

A clean dry 500 mL 3 neck round bottom flask was fit with a coldfinger condenser with vent going to a nitrogen bubbler. A magnetic stir bar, septum, a ¼ inch stainless steel thermocouple were added, and the flask was placed in an oil bath. The flask was charged sequentially with 0.102 mols isopropylborate (19.76 g), 200 mLs of toluene and 0.100 mols of catechol (11.01 g). This mixture was heated to 50° C. to yield a homogeneous solution before adding a solution of 0.100 mols 2-(methylamino)ethanol (7.51 g) and 100 mLs toluene slowly over 1 hour yielding a thick white slurry. The white slurry was allowed to stir at 50° C. for 1 hr and then cooled to room temperature. Vacuum filtration, washing with 50 mLs toluene and drying for 4 hrs yielded 10.78 g (55.9% yield) of white powder SpiroCAT. The filtrate and wash was concentrated under vacuum at 50° C. and 25 mmHg yielding 7.45 g of a tan colored flaky solid (tan color due to unreacted amino-alcohol by ¹H-NMR).

Approximately 42% unreacted IPB is present in the ¹¹B-NMR of the slurry before filtration. Unlike the R-DPP ethylene glycol Spiroborate, the SpiroCAT made in this manner requires heat and azeotropic distillation of isopropanol (IPA) and toluene to drive it to completion. Filtering the reaction before distillation removes some of the IPB, creating an excess of amino-alcohol that stays behind after distillation.

### Example T2

### SpiroCAT in Cyclohexane

Toluene has been a difficult solvent to remove from these spiroborate reactions. Cyclohexane has a BP of 81° C. compared to toluene at 110° C. Both solvents form an azeotrope with IPA.

A clean dry 1 L 3 neck round bottom flask was fit with a coldfinger condenser vented to a nitrogen bubbler, a magnetic stir bar, septum, and a ¼ inch stainless steel thermocouple. The flask, placed in an oil bath, was charged sequentially with 0.200 mols of catechol (22.02 g), 0.204 mots isopropylborate (IPB, 39.52 g) and 400 mLs of toluene This mixture was heated to 50° C. to yield a homogeneous solution before adding a solution of 0.200 mols 2-(methylamino)ethanol (15.02 g) and 200 mLs toluene slowly over 1 hour yielding a thick white slurry. The white slurry was allowed to stir at 50° C. for 1 hr and then cooled to room temperature.

The mixture was concentrated under vacuum at 50-60° C. at 560 mmHg to remove 200 g of solvent. The ¹¹B-NMR showed IPB was still present in the mixture. ¹H-NMR of the distillate resulted in only 55% of the theoretical amount of IPA that should be removed. 250 mL of cyclohexane was back added and the mixture was distilled a second time (to dryness) at the same temperature and vacuum. The white powder was washed with cyclohexane and dried over night yielding 35.98 g, 93.4% yield. The cyclohexane wash contained IPB. While the cyclohexane was easier to remove it did not remove IPA and excess IPB as well as toluene.

### Example P3

### SpiroEA

### CAS Name: Ethanamine, 2-(1,3,2-benzodioxaborol-2-yloxy)-

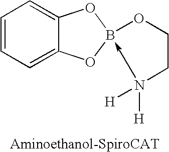

A clean dry 500 mL 3 neck round bottom flask was fit with a coldfinger condenser with vent going to a nitrogen bubbler. A magnetic stir bar, septum, a ¼ inch stainless steel thermocouple were added, and the flask was placed in a water bath. The flask was charged sequentially with 0.102 mols isopropylborate (19.76 g), 200 mLs of toluene and 0.100 mols of catechol (11.01 g). This mixture was heated to 30° C. for 30 mins to yield a homogeneous solution before adding a solution of 0.100 mols ethanolamine (6.11 g) and 100 mLs toluene slowly over 1 hour yielding a thick white slurry. There was an exotherm of 3° C. during addition. The slurry was allowed to stir at room temperature for 1 hr and then vacuum filtered and dried overnight to yield 16.86 g white powder (94% yield).

### Example P4

### 2-(methylamino)ethanol 4-tert-butyl catechol spiroborate (tert-butyl SpiroCAT)

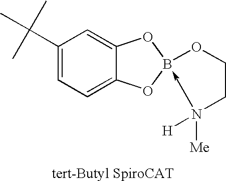

A clean dry 500 mL 3 neck round bottom flask was fit with a coldfinger condenser with vent going to a nitrogen bubbler. A magnetic stir bar, septum, a ¼ inch stainless steel thermocouple were added, and the flask was set in a water bath. The flask was charged sequentially with 0.102 mols isopropylborate (19.76 g), 200 mLs of toluene and 0.100 mols of 4-tert-butyl catechol (16.62 g). This mixture was stirred at room temperature for 30 mins to yield a homogeneous solution before adding a solution of 0.100 mols 2-(methylamino)ethanol (7.51 g) and 100 mLs toluene slowly over 1 hour yielding a thick white slurry. There was an exotherm of 10° C. during addition. An off-white slightly tan precipitate “spiroborate” is formed during addition. The slurry was allowed to stir at room temperature for 1 hr and then concentrated on the rotovap at 50° C. and 25 mmHg. The tacky solids were then redissolved in toluene and vacuum filtered to yield 16.46 g of a white powder (89.51% yield).

¹¹B-NMR: 8.0 ppm.

### Example P5

### SpiroDIME from N,N-dimethylethanolamine and catecholborane

### CAS Name: Ethanamine, 2-(1,3,2-benzodioxaborol-2-yloxy)-N,N-dimethyl-

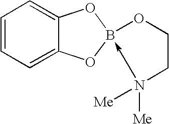

A clean dry 500 mL 3 neck round bottom flask was fit with a coldfinger condenser with vent going to a nitrogen bubbler. A magnetic stir bar, septum, a ¼ inch stainless steel thermocouple were added, and the flask was placed in a water bath. The flask was charged sequentially with 0.102 mols isopropylborate (19.76 g), 200 mLs of toluene and 0.100 mols of catechol (11.01 g). This mixture was held at 30° C. for 30 mins to yield a homogeneous solution before adding a solution of 0.100 mols N,N-dimethylethanolamine (8.91 g) and 100 mLs toluene slowly over 1 hour yielding a thick white slurry. There was an exotherm of 4° C. during addition. The slurry was allowed to stir at room temperature for 1 hr and then vacuum filtered and dried for 4 hrs to yield 17.20 g of a white powder (93.5% yield).

¹¹B-NMR: 11.8 ppm.

### Example P6

### SpiroPCAT

### CAS Name: Pyridine, 2-[(1,3,2-benzodioxaborol-2-yloxy)methyl]-

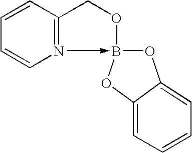

This spiroborate was prepared by reducing 2-pyridine carboxaldehyde with catechol borane (CATB) in toluene. A clean dry 500 mL 3 neck round bottom flask was fit with a coldfinger condenser with vent going to a nitrogen bubbler, a magnetic stir bar, 60 mL addition funnel, a ¼ inch stainless steel thermocouple and placed in an ice-water bath. The flask was charged with 0.084 mols (9.0 g) of 2-pyridine carboxaldehyde and 300 mL of toluene resulting in an intense yellow solution. A solution of 0.084 mols (10.0 g) of CATB and 50 mLs toluene was added over 1 hr maintaining a reaction temperature of 0 to 5° C. Upon addition of CATB a precipitate formed which eventually settled out as a red oily solid that was difficult to stir. Both the red oily solids and yellow slurry had the same ¹¹B-NMR at 13 ppm. The mixture was concentrated under reduced pressure at 70° C. and 25 mmHg resulting in a red oil. The bath was turned off and the flask was allowed to rotate under vacuum as the reaction slowly dropped to room temperature. This yielded 16.25 g (85.53% yield) of reddish-brown needle crystals with some oily spots on the bottom of the flask. Proton NMR of the product showed a trace amount of unreacted aldehyde, toluene and another unknown impurity.

¹¹B-NMR: 13 ppm.

### Example T3

### Reduction of Acetophenone with DEANB and SpiroCAT

Reduction of 0.05 mols of acetophenone with 0.05 mols of DEANB and 5 mol % SpiroCAT in 10 mL THF at room temperature was complete in 1 hr. Without SpiroCAT this reduction takes 4 hrs at 50° C. Using DEANB with 5 wt % DMS, reduction takes 3 hrs at 50° C. Completion of reaction was determined by FT-IR analysis of the carbonyl acetophenone stretch at 1690 cm⁻¹.

### Example T4

### Reduction of Heptane Nitrile with DEANB with and without SpiroCAT

Reduction of 0.05 mols of heptane nitrile with 0.05 mols DEANB in 10 mL THF was done at 50° C. for 24 hrs with and without 5 mol % SpiroCAT. Samples were analyzed by GC to identify the rate and completion of the reaction. At 6 hrs the reaction is 33.9% complete without SpiroCAT and 74.5% complete with SpiroCAT. At 24 hrs the reaction is 79.4% without SpiroCAT and 89.7% with SpiroCAT. While the reduction of heptane nitrile is still slow, there is a significant increase in rate when SpiroCAT is used.

### Reduction Examples 1 to 17

(R=reference examples)

The reduction of ethyl butyrate with DEANS was carried out by addition of DEANB containing an additive to the ester (1:1 mole ratio of borane to ester) at the selected temperature. Reactions were monitored by IR spectroscopy observing the disappearance of the carbonyl stretch. The results with a number of additives at 50° C. are shown in Table 1.

Table 1 shows the acceleration of ethyl butyrate reduction by DEANS with oxazaborolidines as acceleration agents. A dramatic increase in reduction rate was observed with (R)-MeCBS. With this positive results, the reduction of ethyl butylate was selected for further study with other additives, see Table 1.

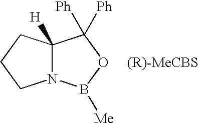

An acceleration in rate was also seen with other oxazaborolidines derived from aminoalcohols. The acceleration agent can be formed in situ from an amino alcohol and the borane (BH₃, examples 6 and 7).

An acceleration was even seen when using an aminodialkoxyborate, DMABO2, demonstrating that the nitrogen atom is not required to be part of a ring.

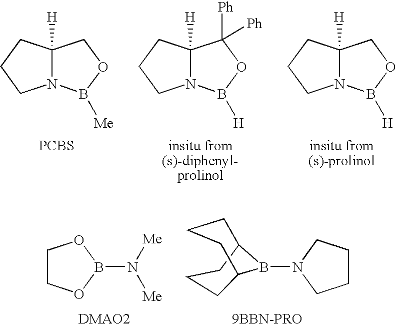

A bicyclic aminoborane was prepared from 9-borabicyclo[3.3.1]nonane and pyrrolidine, dubbed 9BBN-PRO. This compound was not as effective for the ester reduction.

The spiroborate compounds derived from secondary aminoalcohols show the best results thus far. The compounds shown with the acronym of SpiroMO and SpiroCAT decrease the reduction time of ethyl butyrate to 4-5 h at 20° C. The advantage of SpiroCAT over SpiroMO is that the amino alcohol is inexpensive and for an ester or amide reduction a chiral catalyst is not necessary.

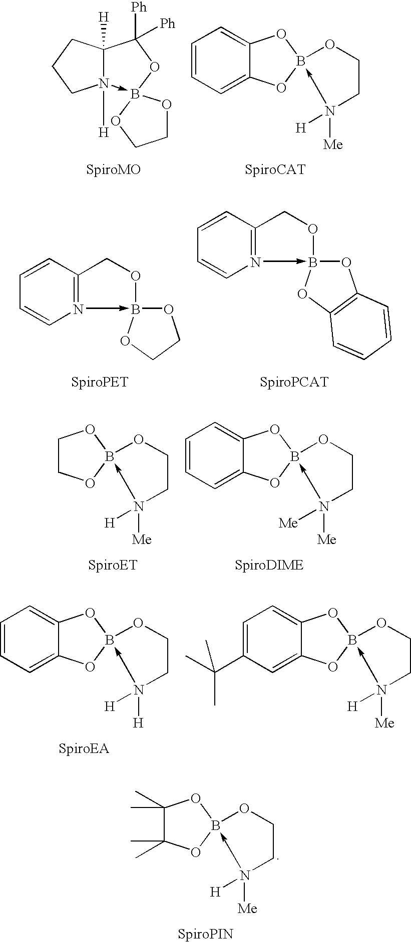

TABLE 1
Ethyl Butyrate Reduction with DEANB
(1:1 ratio of ester:amine borane) in THF

| | | Rxn Temperature | |
|---|---|---|---|
| Example | Additive | (° C.) | Time (hrs) |
| 1 | 5 mol % (R—)Me-CBS | 50 | 1 |
| 2 | 5 mol % (R)—Me-CBS | 20 | 7 |
| 3 | 10 mol % (R)—MeCBS | 20 | 8 |
| 4 | 10 mol % PCBS | 20 | >24 |
| 5 | 10 mol % DMABO2 | 20 | 20 |
| 6 | 10 mol % (S)- | 20 | 20 |
| | diphenylprolinol | | |
| 7 | 10 mol % (S)-Prolinol | 20 | 20 |
| 8 | 10 mol % 9BBN-PRO | 20 | >24 |
| 9 | 10 mol % SpiroMO | 20 | 4.5 |
| 10 | 10 mol % SpiroCAT | 20 | 5 |
| 11 | 10 mol % SpiroPIN | 20 | <18 |
| 12 | 10 mol % SpiroPCAT | 20 | >24 |
| 13 | 10 mol % SpiroET | 20 | 14 |
| 14 | 10 mol % SpiroDIME | 20 | >72 |
| 15 | 10 mol % SpiroEA | 20 | >24 |
| 16 | None | 85 | 9 |
| 17 | None | 50 | >98 |

The compound with a pyridine nitrogen coordination to boron, (SpiroPCAT) and the tertiary amine coordinating to boron (SpiroDIME) are not so effective as catalysts, implying that the amine hydrogen may play a role in the reaction. However, SpiroEA derived from the primary amine, ethanolamine, does not effectively catalyze the ester reduction.

### Examples 18 to 27

Table 2 lists results of additives in the reduction of ethyl benzoate. Table 3 demonstrates the accelerated reduction of N,N-dimethylacetamide by DEANB with oxazaborolidines and other boron compounds as acceleration agents.

TABLE 2
Ethyl Benzoate Reduction with DEANB
(1:1 ratio of ester:amine borane) in THF

| | | Rxn Temperature | |
|---|---|---|---|
| Example | Additive | (° C.) | Time (hrs) |
| R18 | None | 85 | >28 |
| 19 | 1.6 mol % (R)—MeCBS | 20 | 26 |
| 20 | 10 mol % (R)—MeCBS | 20 | 54 |
| 21 | 10 mol % SpiroCAT | 20 | >72 |

TABLE 3
N,N-Dimethylacetamide Reduction by DEANB (1:1.67) in THF

| | | Rxn | |
|---|---|---|---|
| | | Temperature | |
| Example | Additive | (° C.) | Time (hrs) |
| 22 | none | 50 | 6 |
| 23 | 2 mol % (R)—MeCBS | 50 | 1 |
| 24 | 10 mol % (R)—MeCBS | 20 | 1.5 |
| 25 | 5 mol % (R)—MeCBS | 20 | 2 |
| 26 | 10 mol % PCBS | 20 | 4 |
| 27 | 10 mol % SpiroCAT | 20 | 2 |

While the present invention is described herein with reference to illustrated embodiments, it should be understood that the invention is not limited to these examples. Therefore, the present invention is limited only by the claims attached herein.

1. A process for the accelerated reduction of organic substrates, selected from the group consisting of ester, amides, nitriles, acids, ketones, imines or mixtures thereof, by reacting with an amine borane, sulfide borane or ether borane complex as a borane source in the presence of organic accelerator compounds containing both Lewis acidic and Lewis basic sites in their structure, of which the Lewis acidic site can coordinate with the carbonyl or nitrile or imine group of the substrate and the Lewis basic site can coordinate with the borane.

2. A process as claimed in claim 1, wherein esters, acids and ketones are reduced to give alcohols, and amines, nitriles and imines are reduced to give amines.

3. A process as claimed in claim 1, wherein the amine borane, the sulfide borane and the ether borane are derived from amines, sulfides and ethers which conform to the formulae 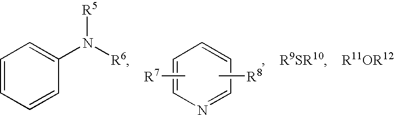
wherein R⁵-R¹² independently are C₁₋₆-alkyl, phenyl, or in which each two of R⁵ and R⁶, R⁹ and R¹⁰, R¹¹ and R¹² independently can together form an C₄₋₆-alkylene group, and R⁵-R¹² can be substituted by halogen and R⁷ and R⁸ can also be hydrogen.

4. A process as claimed in claim 3, wherein the amine borane is N,N-diethylaniline (DEANB), the sulfide borane is dimethylsulfide borane (DMSB), and the ether borane is borane tetrahydrofuran (BTHF) or borane-2-methyltetrahydrofuran.

5. A process as claimed in claim 1, wherein the organic substrate contains 4 to 30 carbon atoms.

6. A process as claimed in claim 5, wherein the organic substrate contains one or more of alkyl, aryl, aralkyl, alkaryl, heterocycloalkyl and heteroaryl groups besides the ester, amide, nitrile acid, keto or imino functional group and may contain other functional groups not reduced by borane.

7. A process as claimed in claim 5, wherein the esters, amides, nitriles, acids, ketones and imines conform to the formulae R¹—C(═O)—OR² R¹—C(═O)—NR³R⁴ R¹—CN R¹—COOH R¹—C(═O)—R² R¹R²C═NH R¹R²C═NR³
wherein
R¹-R⁴ independently are C₁₋₁₂-alkyl, C₆₋₁₂-aryl, C₇₋₁₂-aralkyl, C₇₋₁₂ alkaryl which can be substituted with other functional groups not reduced by borane.

8. A process as claimed in claim 1, wherein the organic accelerator compound contains a structural element of the formula N—B or is an oxazaborolidine or cyclic compound containing a structural element of the formula N—B—O where N— and O— are connected by a carbon chain.

9. A process as claimed in claim 1, wherein the organic accelerator compound is a spiroborate compound containing a structural element of one of the following formulae 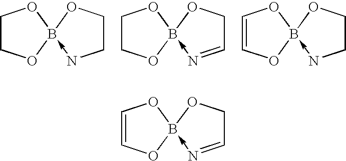
in which the rings can contain 5, 6 or 7 elements.

10. A process as claimed in claim 8, wherein the organic accelerator compound has one of the following general formulae 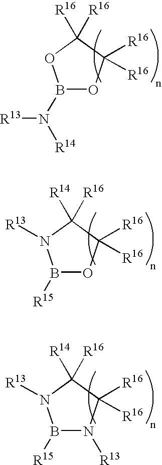
wherein
R¹³, R¹⁴, R¹⁵, R¹⁶ at each position independently are hydrogen, C₁₋₁₂-alkyl, C₆₋₁₂-aryl, C₇₋₁₂-aralkyl, C₇₋₁₂-alkaryl, wherein R¹³ and R¹⁴ or wherein R¹³ and R¹⁵ can together form a cyclic residue, with the proviso that not more than 4 residues R¹⁶ are different from hydrogen,
n is 1, 2 or 3.

11. A process as claimed in claim 10, wherein the oxazaborolidine compound is selected from the group consisting of

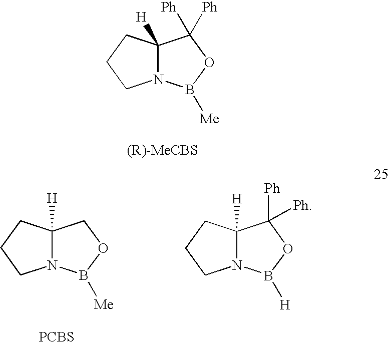

12. A process as claimed in claim 9, wherein the spiroborate compound is selected from the group consisting of

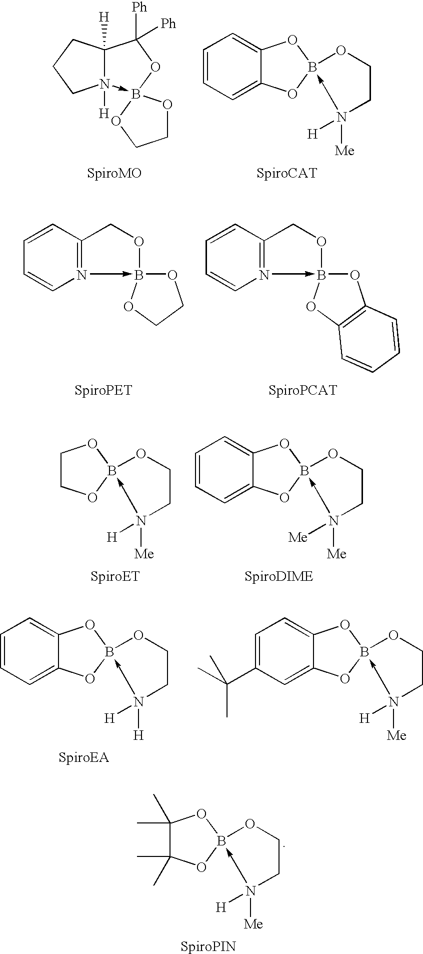

13. A process as claimed in claim 1, wherein the amount of accelerator compound, based on the amine borane, sulfide borane or ether borane is 0.01 to 100 mol-%.

14. A composition for the accelerated reduction of organic substrates, selected from the group consisting of esters, amides, nitriles, acids, ketones, imines or mixtures thereof comprising at least one amine borane, sulfide borane or ether borane complex as a borane source and at least one organic accelerator compound containing both Lewis acid acidic and Lewis basic sites in their structure, of which the Lewis acidic site can coordinate with the carbonyl or nitrile or imino group of a substrate and the Lewis basic site can coordinate with the borane.

15. An organic accelerator compound as defined in claim 10.

16. An organic accelerator compound as defined in claim 12.
